(19) United States
(12) Patent Application Publication
(10) Pub. No.: US 2011/0014666 A1
(43) Pub. Date: Jan. 20, 2011

(54) POLYPEPTIDE HAVING GLYOXALASE III ACTIVITY, POLYNUCLEOTIDE ENCODING THE SAME AND USES THEREOF

(75) Inventors: Francois Voelker, Montrond Les Bains (FR); Philippe Soucaille, Deyme (FR)

Correspondence Address: Baker Donelson Bearman, Caldwell & Berkowitz, PC, 920 Massachusetts Ave, NW, Suite 900, Washington, DC, 20001, US

(73) Assignee: Metabolic Explorer, Saint Beauzire (FR)

(21) Appl. No.: 12/933,199

(22) PCT Filed: Mar. 16, 2009
(86) PCT No.: PCT/EP09/53093

§ 371 Date: Sep. 17, 2010

(30) Foreign Application Priority Data
Mar. 18, 2008 (EP) PCT/EP2008/053224

(51) Int. Cl. C12P 7/56; C12N 9/88; C07H 21/00; C12N 15/63; C12N 1/00; C12N 1/21; C12N 1/19; C12N 1/15; C12P 7/18; C12P 7/28

(52) U.S. Cl. 435/139; 435/232; 536/23.2; 435/320.1; 435/243; 435/252.3; 435/254.2; 435/254.11; 435/252.32; 435/252.35; 435/252.31; 435/252.33; 435/158; 435/150

(57) ABSTRACT

The present invention relates to a novel polypeptide having the enzymatic activity of conversion of methylglyoxal to lactic acid in a single step (known as glyoxalase III activity), a polynucleotide having a nucleotide sequence encoding such polypeptide and uses thereof.

The invention relates to the modulation of the glyoxalase III activity in a microorganism by varying the expression level of the polynucleotide coding for such polypeptide.

The invention also relates to the production of commodity chemicals, especially 1,2-propanediol, acetol, and lactic acid by fermenting microorganisms wherein their glyoxalase III activity is modulated.

### FIELD OF THE INVENTION

The present invention relates to a novel polypeptide having the enzymatic activity of conversion of methylglyoxal to lactic acid in a single step (known as glyoxalase III activity), a polynucleotide having a nucleotide sequence encoding such polypeptide and uses thereof.

More particularly, the invention relates to the modulation of the glyoxalase III activity in a microorganism by varying the expression level of the polynucleotide coding for such polypeptide.

The present invention also relates to the production of commodity chemicals, especially 1,2-propanediol, acetol, and lactic acid by fermenting microorganisms wherein their glyoxalase III activity is modulated.

### PRIOR ART

Catabolism of glucose can result in lactate formation in several microorganisms, generally under conditions of fermentation. D- or L-lactate can be formed depending on the organisms: D-lactate is formed in E. coli together with other products during mixed acid fermentation, whereas D- or L-lactate can be produced by fermentation of lactic acid bacteria. The production pathway is derived from the glycolysis pathway leading from glucose to pyruvate. Pyruvate can be reduced by a single reaction into lactate by a soluble lactate dehydrogenase dependent on reduced nicotinamide adenine dinucleotide (NADH) as co-factor. In E. coli, the lactate dehydrogenase coded by the ldhA gene is specific for D-lactate (Clark, 1997). In lactic acid bacteria, lactate dehydrogenases specific for D- or L-lactate can be found (D-lactate for Lactobacillus delbrueckii, L-lactate for Lactobacillus helveticus, see for example Garvie, 1980).

In several organisms, another pathway can be responsible for lactate production. This pathway is called methylglyoxal bypass as it could serve as an alternative to the downstream part of the glycolysis pathway converting the triose glyceraldehyde-3-phosphate (GA3P) into pyruvate (Cooper, 1984). Methylglyoxal bypass starts from the second triose phosphate produced by the cleavage of fructose-1,6-bisphosphate, dihydroxyacetone phosphate (DHAP). DHAP is converted to methylglyoxal (MG) by methylglyoxal synthase. MG, which is a toxic compound for the cell is then converted to D- or L-lactate by different systems and the lactate produced can be further transformed into pyruvate by D- or L-lactate dehydrogenases. These enzymes, in contrast to fermentative lactate dehydrogenases, are flavin-linked membrane-bound proteins that are activated only under aerobic conditions (Garvie, 1980). D- and L-lactate dehydrogenases are coded respectively by the dld and lldD (or lctD) genes in E. coli (Rule et al, 1985, Dong et al, 1993).

The routes for catabolism of methylglyoxal have been investigated in bacteria (Ferguson et al, 1998) to understand the detoxification of this compounds but also for purposes of production of 1,2-propanediol. Three pathways that can lead to the production of lactate from methylglyoxal have been identified in E. coli:

The first one is the glutathione dependent glyoxalase I-II system (encoded by gloA and gloB genes) which converts methylglyoxal into D-lactate in two steps.

The second is the glutathione independent glyoxalase III enzyme which catalyses the conversion of methylglyoxal into D-lactate in one step.

The third system is the degradation of methylglyoxal by methylglyoxal reductases, resulting either in acetol or in D- or L-lactaldehyde. L-lactaldehyde can be further converted to L-lactate by the action of aldehyde dehydrogenases e.g. by the enzymes encoded by the aldA or aldB genes (Grabar et al, 2006).

The glyoxalase III system has been less extensively studied than the glyoxalase I-II system. The enzyme glyoxalase III was first mentioned in E coli by Misra et al in 1995 and purified. This enzyme is significantly different from glyoxalase I as it has different properties and is able to catalyse the conversion of methylglyoxal in D-lactate in a single step, independently of gluthatione. This enzyme is later mentioned in several reports (MacLean et al, 1998, Okado-Matsumoto and Fridovich, 2000, Benov et al 2004) to have a higher activity than glyoxalase I or glyoxalase II in E. coli. Before this day, the amino-acid sequence of glyoxalase III had not been determined and the gene coding for this enzyme was unknown.

1,2-propanediol or propylene glycol, a C3 dialcohol, is a widely-used chemical. It is a component of unsaturated polyester resins, liquid detergents, coolants, anti-freeze and de-icing fluids for aircraft. Propylene glycol has been increasingly used since 1993-1994 as a replacement for ethylene derivatives, which are recognised as being more toxic than propylene derivatives.

1,2-propanediol is currently produced by chemical means using a propylene oxide hydration process that consumes large amounts of water. Propylene oxide can be produced by either of two processes, one using epichlorhydrin, and the other hydroperoxide. Both routes use highly toxic substances. In addition, the hydroperoxide route generates by-products such as tert-butanol and 1-phenyl ethanol. For the production of propylene to be profitable, a use must be found for these by-products. The chemical route generally produces racemic 1,2-propanediol, whereas each of the two stereoisomers (R)1,2-propanediol and (S)1,2-propanediol are of interest for certain applications (e.g. chiral starting materials for specialty chemicals and pharmaceutical products).

Acetol or hydroxyacetone (1-hydroxy-2-propanone) is a C3 keto alcohol. This product is used in vat dyeing process in the textile industry as a reducing agent. It can advantageously replace traditional sulphur containing reducing agents in order to reduce the sulphur content in wastewater, harmful for the environment. Acetol is also a starting material for the chemical industry, used for example to make polyols or heterocyclic molecules. It possesses also interesting chelating and solvent properties.

Acetol is currently produced mainly by catalytic oxidation or dehydration of 1,2-propanediol. New processes starting from renewable feedstocks like glycerol are now proposed (see DE4128692 and WO 2005/095536). Currently, the production cost of acetol by chemical processes reduces its industrial applications and markets.

The disadvantages of the chemical processes for the production of 1,2-propanediol and acetol make biological synthesis an attractive alternative. The only economically feasible biological route utilizes the natural production pathway for 1,2-propanediol from common sugars (e.g. glucose or xylose) found in some microorganisms. Glucose is metabolized through the glycolysis pathway followed by the methylglyoxal bypass and MG can be reduced either to lactaldehyde or to acetol. These two compounds can then undergo a second reduction reaction yielding 1,2-propanediol. This route is used by natural producers of (R)-1,2-propanediol, such as Clostridium sphenoides and Thermoanaerobacter thermosaccharolyticum. Clostridium sphenoides has been used to produce 1,2-propanediol at a titer of 1,58 g/l under phosphate limited conditions (Tran Din and Gottschalk, 1985). Thermoanaerobacter thermosaccharolyticum has also been investigated for the production of 1,2-propanediol (Cameron and Cooney, 1986, Sanchez-Rivera et al, 1987). The best performances obtained were a titer of 9 g/l and a yield from glucose of 0.2 g/g. However, the improvement of the performances obtained with these organisms is likely to be limited due to the shortage of available genetic tools. The same synthesis pathway is functional in E. coli and several investigations have been done by the group of Cameron (Cameron et al, 1998, Altaras and Cameron, 1999, Altaras and Cameron, 2000) and the group of Bennett (Huang et al, 1999, Berrios-Rivera et al, 2003) for the production of 1,2-propanediol in this organism. The best results obtained by the group of Cameron are production of 1.4 g/l 1,2-propanediol in anaerobic flask culture with a yield of 0.2 g/g of glucose consumed. When extrapolated to an anaerobic fed-batch fermenter, the production was 4.5 g/l 1,2-propanediol with a yield of 0.19 g/g from glucose, far from the theoretical evaluation of Cameron et al. Results obtained with the same approach but with lower titers and yields are also described in the patents U.S. Pat. No. 6,087,140, U.S. Pat. No. 6,303,352 and WO 98/37204. The group of Bennett obtained similar results in flask cultures under anaerobic conditions with a titer of 1.3 g/l and a yield of 0.12 g/g whereas microaerobic cultures gave a titer of 1.4 g/l with a yield of 0.13 g/g.

An alternative method to obtain a strain producing 1,2-propanediol and/or acetol is to direct the evolution of an “initial strain” towards a state where the “evolved strain” produces the desired compound with better characteristics. This procedure to obtain an “evolved strain” of micro-organism for the production of 1,2-propanediol is described in the patent application WO 2005/073364. This evolution process and the following step of fermentation are preferentially performed under anaerobic conditions. This technology is a clear improvement over the prior art. A 1,2-propanediol titer of 1.8 g/l was obtained, with a yield of 0.35 g/g of glucose consumed. An improvement of this method was described in the patent applications WO 2008/116852 and WO 2008/116849 where an evolved strain obtained according to the procedure described above is furthermore modified to obtained respectively a better 1,2-propanediol producer or a better acetol producer. With such a method, yields higher than 1 mol 1,2-propanediol per mol of glucose consumed (0.42 g/g) can be obtained. The same performances can be obtained by a strain building strategy relying only on rational metabolic engineering as described in WO 2008/116848 for production of 1,2-propanediol or WO 2008/116851 for production of acetol.

D- or L-lactate appeared to be common contaminants of the 1,2-propanediol production processes. The competing pathways have been identified as the fermentative lactate dehydrogenase pathway and the glyoxalase I-glyoxalase II pathway. These pathways have been targeted through deletion of the ldhA gene in E. coli (Berrios-Rivera et al, 2003) or the deletion of both the ldhA and gloA genes in E. coli (Altaras and Cameron, 2000) and the yield in 1,2-propanediol from glucose shown to increase accordingly. Another pathway for the production of L-lactate through the oxidation of L-lactaldehyde has been eliminated in WO 2005/073364 with the deletion of the aldA and aldB genes in addition to the ldhA and gloA genes. However, even with these 4 deletions, lactate was still produced under certain conditions. Glyoxalase III appeared to be responsible for this production, impacting both the yield and selectivity of the 1,2-propanediol production process.

Lactic acid or lactate and its derivatives have a wide range of applications in the food, pharmaceutical, leather and textile industries. Recently, polylactic acid (PLA) has been developed as a renewable, biodegradable and environmentally friendly plastic and therefore, the demand for lactate is expected to expand. Lactate can be produced either by a chemical synthesis or by a biological process. However, only a biological process is able to produce the desired stereoisomer, D- or L-lactate with high optical purity, which is an important characteristic for many of its end uses. Physical properties and biodegradation rate of PLA can be controlled by manipulating the ratio of the chiral substrates, D- and L-lactate. Therefore, availability of biological processes for the production of optically pure D- and L-lactate is a prerequisite for high quality polymer synthesis.

Lactic acid bacteria are natural producers of lactate and some can be found to be specific for the D- or L-form. These bacteria have been traditionally used for the production of lactate as specialty chemical (e.g. in US 2004/0005677). However, with the emergence of lactate as commodity chemical for PLA synthesis, more efficient and cost-effective processes are needed. Alternative biocatalysts able to growth in mineral salt medium and to use a range of different sugar substrates are investigated. Yeasts and E. coli combine these characteristics with the availability of a wide range of genetic tools for metabolic engineering. Use of these catalysts for the production of lactic acid has been described in WO 03102201, WO 03102152 and US 2005/0112737 for yeast strains and in EP 1760156 and WO 2005/033324 for E. coli strains.

The synthesis of D- or L-lactate in microorganisms relies on the reduction of pyruvate produced by the catabolism of sugars by NADH-dependent lactate dehydrogenases. Conditions for efficient conversion are generally achieved under anaerobiosis where a large pool of NADH co-factor is available. Lactic acid bacteria can be selected for homofermentative metabolism yielding lactate as the only fermentation product. This is not the case with yeast or E. coli and other fermentation products like ethanol, acetate, formate or succinate have to be removed. This can be achieved by genetic engineering with deletion of the corresponding genes.

In the recent years, metabolic engineering of E. coli for production of optically pure D-lactate has been investigated. Chang et al. (1999) used a pta mutant defective in the acetate production pathway and showed that the carbon flux can be redirected toward D-lactate production by use of the native lactate dehydrogenase. However, part of the carbon flux is still diverted toward the synthesis of by products, especially succinate. Improved biocatalyst have been developed by Zhou et al (2003a) with inactivation of genes encoding acetate kinase (ackA), fumarate reductase (frdABCD), alcool/aldehyde dehydrogenase (adhE) and pyruvate formate lyase (pflAB) in order to prevent the synthesis of acetate, succinate, ethanol and formate respectively. These modifications resulted in a strain able to produce D-lactate with 98% chemical purity and more than 99% optical purity. The same approach has been used by the same authors (Chang et al, 1999, Zhou et al, 2003b) and by Dien et al (2001) to engineer E. coli for the production of L-lactate by additionally replacing the ldhA gene by a lactate dehydrogenase gene from an other organism (Lactobacillus casei, Streptococcus bovis or Pediococcus acidilactici) and specific for the production of L-lactate. For a strain with deletions in ackA, frdABCD, adhE and pflAB genes, Zhou et al (2003b) reported a production of L-lactate with 98% chemical purity and more than 99% optical purity. Such strategies are also described in EP 1760156.

In the D- and L-lactate producers developed by the team of Ingram (Zhou et al, 2003a and b), a weak chiral contamination was observed during the development of the strains for better lactate production. This contamination was shown to come from the production of either D- or L-lactate in the methylglyoxal bypass (Grabar et al, 2006), as discussed above. This contamination can be prevented by engineering a strain with a deletion in the mgsA gene. This work highlighted the methylglyoxal bypass as an alternative non-fermentative pathway for the production of lactate. This alternative production pathway has not yet been used to build a process for the production of lactate.

### DESCRIPTION OF THE INVENTION

The present invention concerns an isolated polypeptide having a glyoxalase III enzymatic activity comprising the sequence of SEQ ID NO 1, a fragment or homologous sequence thereof

The invention also provides a polynucleotide comprising a sequence coding for said polypeptide. Inventors report the identification of a gene from E. coli encoding a protein having a glyoxalase III activity. This gene was previously known as the yedU gene (also known as hchA) coding for Hsp31, a homodimeric protein (Sastry et al, 2002). This protein has been later purified, crystallized and its structure resolved by different groups (Lee et al, 2003, Quigley et al, 2003 and Zhao et al, 2003). Several functions have been associated with Hsp31: molecular chaperone active in the management of protein misfolding (Malki et al, 2003), aminopeptidase of broad specificity (Malki et al, 2005) and another potential function linked to the 2-His-1-carboxylate motif able to coordinate a metal ion and present in several dioxygenases and hydroxylases (Zhao et al, 2003). No association of Hsp31 with a glyoxalase III activity has never been reported in the literature.

The invention is furthermore related to an expression cassette comprising said polynucleotide under the control of regulatory elements functional in a host cell and to a transformation vector comprising said cassette or said polynucleotide.

The invention also provides a modified microorganism having modulated glyoxalase III enzymatic activity wherein activity of the polynucleotide of the invention is attenuated or enhanced.

It is one object of the present invention to use the new knowledge of a gene encoding for the glyoxalase III protein from E. coli to design a microorganism having an attenuated glyoxalase III enzymatic activity. This microorganism is able to convert glucose to 1,2-propanediol or acetol, with an improved yield and with a better selectivity (i.e. less by-products) compared to the already-known processes.

In certain embodiments of the invention, microorganisms according to the invention with attenuated glyoxalase III activity are further modified to enhance the production of 1,2-propanediol and/or acetol. Additionally, a method for preparing 1,2-propanediol and/or acetol wherein said microorganisms are grown in appropriate growth medium and 1,2-propanediol and/or acetol is recovered is provided.

It is also an object of the present invention to use the new knowledge of the gene encoding for the glyoxalase III protein from E. coli to design a microorganism useful for the production of lactate. Overexpression of said gene coding for glyoxalase III provides a strain able to produce lactate under fully aerobic conditions, therefore increasing the productivity of the process. A method for preparing lactate wherein said microorganisms are grown in appropriate growth medium and lactate is recovered is provided.

The invention is also related to a method for modulating the glyoxalase III enzymatic activity in a microorganism, wherein activity of the polypeptide of the invention is enhanced or attenuated in said microorganism.

### DETAILED DESCRIPTION OF THE INVENTION

The present invention is related to an isolated polypeptide having a glyoxalase III enzymatic activity comprising the sequence of SEQ ID No1, a fragment or homologous sequence thereof.

As used herein, the following terms may be used for interpretation of the claims and specification.

According to the invention, the term “polypeptide” refers to peptide or protein which comprises a sequence of two or more amino-acids linked with peptide bonds.

The term “isolated” refers to a protein or DNA sequence that is removed from at least one component with which it is naturally associated.

The term “glyoxalase III” refers to a polypeptide responsible for an enzyme activity that catalyzes the conversion in a single step of methylglyoxal into D-lactate. Such an enzyme activity was described in E. coli by Misra et al (1995) and methods to measure this enzyme activity were provided.

The terms “enzyme activity” and “enzymatic activity” are used interchangeably and refer to the ability of an enzyme to catalyse a specific chemical reaction, for example the conversion of methylglyoxal in D-lactate for glyoxalase III enzyme activity.

The isolated polypeptide of the present invention can be obtained from microorganisms having glyoxalase III activity, for example by using the purification procedure as described in the following examples. Microorganisms that can be used to isolate the polypeptide include, but are not limited to, E. coli

The term “comprising the sequence of SEQ ID No1” means that the amino-acid sequence of the polypeptide may not be strictly limited to SEQ ID No1 but may contain additional amino-acids. The term “a fragment of SEQ ID No1” means that the sequence of the polypeptide may include less amino-acid than SEQ ID No1 but still enough amino-acids to confer glyoxalase III activity. It is well known in the art that a polypeptide can be modified by substitution, insertion, deletion and/or addition of one or more amino-acids while retaining its enzymatic activity. For example, substitutions of one amino-acid at a given position by a chemically equivalent amino-acid that do not affect the functional properties of a protein are common. For the purposes of the present invention, substitutions are defined as exchanges within one of the following groups:

Small aliphatic, non-polar or slightly polar residues: Ala, Ser, Thr, Pro, Gly

Polar, negatively charged residues and their amides: Asp, Asn, Glu, Gln

Polar, positively charged residues: His, Arg, Lys

Large aliphatic, non-polar residues: Met, Leu, Ile, Val, Cys

Large aromatic residues: Phe, Tyr, Trp.

Thus, changes which result in substitution of one negatively charged residue for another (such as glutamic acid for aspartic acid) or one positively charged residue for another (such as lysine for arginine) can be expected to produce a functionally equivalent product.

The positions where the amino-acids are modified and the number of amino-acids subject to modification in the amino-acid sequence are not particularly limited. The man skilled in the art is able to recognize the modifications that can be introduced without affecting the activity of the protein. For example, modifications in the N- or C-terminal portion of a protein would not be expected to alter the activity of a protein.

The term “homologous” refers to polypeptides submitted to modifications such as defined above while still retaining the original enzymatic activity.

In a specific embodiment of the invention, the polypeptide of the present invention have at least 70% identity with the sequence of SEQ ID No1, preferentially at least 80% identity and more preferentially at least 90% identity.

Methods for determination of the percentage of identity between two protein sequences are known from the man skilled in the art. For example, it can be made after alignment of the sequences by using the software CLUSTALW available on the website http://www.ebi.ac.uk/clustalw/ with the default parameters indicated on the website. From the alignment, calculation of the percentage of identity can be made easily by recording the number of identical residues at the same position compared to the total number of residues. Alternatively, automatic calculation can be made by using for example the BLAST programs available on the website http://www.ncbi.nlm.nih.gov/BLAST/ with the default parameters indicated on the website.

In a specific embodiment of the invention, the polypeptide comprises at least 100 contiguous amino-acids from the sequence of SEQ ID No1, preferentially at least 150, at least 200, at least 250 or more preferentially at least 280 contiguous amino-acids of the sequence shown in SEQ ID No1 In another embodiment of the invention, the polypeptide has a polypeptidic sequence strictly identical to the sequence of SEQ ID No1.

The present invention is also related to a polynucleotide comprising a sequence coding for the polypeptide of the invention.

The term “polynucleotide” refer to a polymer of ribonucleotides (or RNA) or to a polymer of deoxyribonucleotides (or DNA), that is single or double-stranded, optionally containing synthetic, non-natural, or altered nucleotide bases. An isolated polynucleotide in the form of DNA may contain one or more segments of synthetic DNA, genomic DNA or cDNA.

The origin of the polynucleotide is not necessarily the organism where the enzymatic activity is originally measured. Hybridization under different conditions of stringency with a probe that comprises the nucleotide sequence of SEQ ID No2 can be used to screen a gene library for such polynucleotides by the man skilled in the art. Detailed protocols for hybridization are disclosed in Sambrook et al (1989).

The sequences of such polynucleotides can be extracted from the databases using for example the BLAST programs defined above and searching for homology with the nucleotide sequence of SEQ ID No2.

Preferred polynucleotides of the present invention are polynucleotides that are at least 80% identical to the nucleotide sequence of SEQ ID No2. More preferred polynucleotides of the present invention are polynucleotides that are at least 90% identical to the nucleotide sequence of SEQ ID No2. Even more preferred polynucleotides of the present invention are polynucleotides that are at least 95% identical to the nucleotide sequence of SEQ ID No2.

In particular, the polynucleotide that comprises the nucleotide sequence of SEQ ID No2 is included in the invention.

The terms “encoding” or “coding” refer to the process by which a polynucleotide, through the mechanisms of transcription and translation, produces an amino-acid sequence. This process is allowed by the genetic code, which is the relation between the sequence of bases in DNA and the sequence of amino-acids in proteins. One major feature of the genetic code is to be degenerate, meaning that one amino-acid can be coded by more than one triplet of bases (one “codon”). The direct consequence is that the same amino-acid sequence can be encoded by different polynucleotides. As an example, polynucleotide sequences derived from SEQ ID No2 by degeneracy of the genetic code can also code for the polypeptide sequence of SEQ ID No1 and are therefore contemplated by the present invention. It is well known from the man skilled in the art that the use of codons can vary according to the organisms. Among the codons coding for the same amino-acid, some can be used preferentially by a given microorganism. It can thus be of interest to design a polynucleotide adapted to the codon usage of a particular microorganism in order to optimize the expression of the corresponding protein in this organism.

The present invention is also related to an expression cassette comprising the polynucleotide of the invention under the control of regulatory elements functional in a host microorganism.

The term “expression” refers to the transcription and translation of a gene sequence leading to the generation of the corresponding protein, product of the gene.

The term “expression cassette” refers to a polynucleotide preferably linked with regulatory elements, such as promoters, enhancers, ribosome binding site or terminator allowing the expression of the gene contained in the polynucleotide inside a suitable host organism. Such regulatory elements can be the own regulatory elements of the gene, but also modified or synthetic elements, to allow a stronger expression of the gene. For example, stronger expression can be obtained by replacing the native promoter of the gene by stronger promoters. For E. coli these promoters are for example: lac promoter, tac promoter, trc promoter and lambda cI promoter. For other organisms, the skilled artisan may be able to choose the more adapted promoter.

The term “host microorganism” refers to a microorganism able to receive foreign or heterologous genes or extra copies of its own genes and able to express those genes to produce an active protein product.

The present invention provides for a transformation vector comprising the polynucleotide or the cassette according to the invention.

The term “transformation” refers to the introduction of new genes or extra copies of existing genes into a host organism. The acquired genes may be incorporated into chromosomal DNA or introduced as extra-chromosomal elements. As an example, in E. coli, a method for transferring DNA into a host organism is electroporation.

The term “transformation vector” refers to any vehicle used to introduce a polynucleotide in a host organism. Such vehicle can be for example a plasmid, a phage or other elements known from the expert in the art according to the organism used. The transformation vector usually contains in addition to the polynucleotide or the expression cassette other elements to facilitate the transformation of a particular host cell. An expression vector comprises an expression cassette allowing the suitable expression of the gene borne by the cassette and additional elements allowing the replication of the vector into the host organism. An expression vector can be present at a single copy in the host organism or at multiple copies.

The present invention also provides for a modified microorganism having modulated glyoxalase III activity, wherein activity of the polypeptide of the invention is attenuated or enhanced.

The term “attenuation of the activity of an enzyme” refers to a decrease of activity of the enzyme of interest, compared to the observed activity in the same microorganism before any modification. The man skilled in the art knows numerous means to obtain this result, and for example:
Introduction of a mutation into the gene, decreasing the expression level of this gene, or the level of activity of the encoded protein.
Replacement of the natural promoter of the gene by a low strength promoter, resulting in a lower expression.
Use of elements destabilizing the corresponding messenger RNA or the protein.
Deletion of the gene if no expression at all is needed.

An “increased enzymatic activity” or an “enhanced enzymatic activity” means that the activity is superior to the original activity measured in the same microorganism before any modification. The corresponding non-modified microorganism is a microorganism having the same characteristics of the modified microorganism except for the enzyme activity under consideration. Advantageously, the enzyme activity is increased by at least 50%, preferably by at least 100%, compared to the native activity of the corresponding non-modified microorganism. A method for measuring glyoxalase III activity is given in Example 1 below.

The microorganism according to the invention is selected among the group consisting of bacteria, yeast and fungi. Preferentially, the bacterium is selected among the group consisting of Enterobacteriaceae, Bacillaceae, Clostridiaceae, Streptomycetaceae and Corynebacteriaceae. More preferentially, the bacterium is selected among the group consisting of Escherichia coli, Bacillus subtilis, Clostridium acetobutylicum and Corynebacterium glutamicum.

It is an object of the present invention to provide a microorganism with attenuated glyoxalase III activity, wherein the expression of the native gene coding for the polypeptide of the invention is attenuated.

The term “attenuation of the expression of a gene” according to the invention denotes the partial or complete suppression of the expression of a gene, which is then said to be “attenuated”. This suppression of expression can be either an inhibition of the expression of the gene, a deletion of all or part of the promoter region necessary for the gene expression, or a deletion in the coding region of the gene. Preferentially, the attenuation of a gene is essentially the complete deletion of that gene, which gene can be replaced by a selection marker gene that facilitates the identification, isolation and purification of the strains according to the invention. A gene is inactivated preferentially by the technique of homologous recombination (Datsenko, K. A. & Wanner, B. L., 2000).

In one embodiment of the invention, the microorganism with attenuated glyoxalase III activity is further modified to enhance production of 1,2-propanediol and/or acetol from a source of carbon.

In a specific embodiment of the invention, in the microorganism according to the invention, some enzyme activities involved either in bypass pathways or by-product formation pathways are attenuated in order to increase the yield of 1,2-propanediol and/or acetol production from a source of carbon:

Attenuation of the enzymatic activities involved in the Entner-Doudoroff pathway, encoded by the genes edd and eda. The Entner-Doudoroff pathway provides an alternative way to degrade glucose to glyceraldehyde-3-phosphate and pyruvate besides glycolysis. The attenuation of the Entner-Doudoroff pathway assures that most or at best all glucose is degraded via glycolysis and is utilized for the production of 1,2-propanediol.

Attenuation of enzymes involved in the conversion of methylglyoxal into lactate: glyoxalase I, encoded by the gloA gene, catalysing the synthesis of lactoyl glutathione from methylglyoxal and lactaldehyde dehydrogenases encoded by the aldA and aldB genes, catalysing the synthesis of (S) lactate from (S) lactaldehyde. Attenuation of these enzymes is intended to spare the precursor methylglyoxal for the synthesis of the desired products.

Attenuation of enzymes involved in the synthesis of by-products such as lactate, ethanol and formate: lactate dehydrogenase, encoded by the gene ldhA, catalysing the synthesis of lactate from pyruvate, alcohol-aldehyde dehydrogenase, encoded by the gene adhE, catalysing the synthesis of ethanol from acetyl-CoA and pyruvate formate lyase, encoded by the genes pflA and pflB, catalysing the synthesis of acetyl-CoA and formate from pyruvate.

Preferentially, at least one of these genes is attenuated.

In another specific embodiment of the invention, the triose phosphate isomerase activity is attenuated. Preferentially, this result is achieved by attenuating the expression of the tpiA gene. More preferentially, the tpiA gene is deleted. The tpiA gene encodes the enzyme ‘triose phosphate isomerase’, which catalyses the conversion of DHAP into glyceraldehyde 3-phosphate. The attenuation of the expression of this gene ensures that half of the glucose metabolized is converted to 1,2-propanediol and/or acetol.

In another specific embodiment of the invention, the glyceraldehyde 3 phosphate dehydrogenase activity is attenuated. The glyceraldehyde 3-phosphate dehydrogenase, also called GAPDH, is one of the key enzymes involved in the glycolytic conversion of glucose to pyruvic acid. The attenuation of the enzyme resulted in the redirection of part of the GA3P toward the synthesis of 1,2-propanediol and/or acetol. The yield of 1,2-propanediol over glucose can then be greater than 1 mole/mole. Advantageously, the activity of the glyceraldehyde 3-phosphate dehydrogenase is about less than 30% of the usual activity of a wild-type GADPH, more preferably less than 10%. Preferentially, the expression of the gapA gene coding for GAPDH is attenuated.

In another embodiment of the invention, in the microorganism according to the invention, the efficiency of the sugar import is increased. A strong attenuation of the expression of the gapA gene resulting in a decrease of the carbon flux in the GAPDH reaction by more than 50% result in the synthesis of less than 1 mole of PEP per mole of glucose imported. PEP is required by the sugar-phosphotransferase system (PTS) normally used for the import of simple sugars into the cell, since import is coupled to a phospho-transfer from PEP to glucose yielding glucose-6-phosphate. Thus reducing the amount of PEP will negatively impact on sugar import.

In a specific embodiment of the invention, the sugar might be imported into the microorganism by a sugar import system independent of phosphoenolpyruvate. The galactase-proton symporter encoded by the gene galP that does not involve phosphorylation can be utilized. In this case, the imported glucose has to be phosphorylated by the glucose kinase activity encoded by the glk gene. To promote this pathway, the expression of at least one gene selected among galP and glk is increased. As a result the PTS becomes dispensable, it can be eliminated by attenuating at least one gene selected among ptsG, ptsH, ptsI or crr. These four genes are coding for the different functional domains of the PTS complex: ptsG codes for the two subunits B and C of the Enzyme II, ptsH codes for the HPr protein, ptsI codes for the Enzyme I and crr codes for the subunit A of the Enzyme II.

In another specific embodiment of the invention, the efficiency of the sugar-phosphotransferase system (PTS) is increased by increasing the availability of the metabolite phosphoenolpyruvate. Due to the attenuation of the gapA activity and of the lower carbon flux toward pyruvate, the amount of PEP in the modified strain of the invention could be limited, leading to a lower amount of glucose transported into the cell.

Various means exist that may be used to increase the availability of PEP in a strain of microorganism. In particular, a mean is to attenuate the reaction PEP→pyruvate. Preferentially, at least one gene selected among pykA and pykF, coding for the pyruvate kinase enzyme, is attenuated in said strain to obtain this result. Another way to increase the availability of PEP is to favour the reaction pyruvate→PEP, catalysed by the phosphoenolpyruvate synthase by increasing the activity of this enzyme. This enzyme is encoded by the ppsA gene. Therefore, preferentially in the microorganism, the expression of the ppsA gene is preferentially increased. Both modifications can be present in the microorganism simultaneously.

In another specific embodiment of the invention, the synthesis of the by-product acetate is prevented by attenuating at least one enzyme involved in its synthesis It is preferable to avoid such acetate synthesis to optimize the production of 1,2-propanediol.

To prevent the production of acetate, advantageously at least one gene selected among ackA, pta and poxB is attenuated. These genes all encode enzymes involved in the different acetate biosynthesis pathways.

Specifically for the production of 1,2-propanediol, it may be advantageous to increase the specific enzyme activities leading to the formation of this compound: methylglyoxal synthase, methylglyoxal reductase and 1,2-propanediol dehydrogenase. Advantageously, the enzyme activities are increased by at least 50%, preferably by at least 100%, compared to the native activity of the corresponding non-modified microorganism.

To obtain the increase of the specific enzymatic activities, the genes coding for these activities are preferentially overexpressed: the mgsA gene, coding for methylglyoxal synthase, yqhD, yafB, ydhF, ycdW, yqhE, yeaE, yghZ, yajO, tas, ydjG, and ydbC, all coding for methylglyoxal reductases, gldA or fucO, coding for 1,2-propanediol dehydrogenase.

The combination of the overexpression of the mgsA, yqhD and gldA genes is preferentially used.

To obtain an overexpression of a gene of interest, the man skilled in the art knows different methods, and for example:

1—Replacement of the native promoter of the gene with a promoter inducing a stronger level of expression of the gene of interest.

2—Introduction into the microorganism of an expression vector carrying and expressing said gene of interest.

3—Introduction of additional copies of the gene of interest into the chromosome of the microorganism.

Another way to obtain an increased enzymatic activity is to introduce into the gene of interest a specific mutation allowing the translation of a gene product presenting a higher activity than the native protein.

Under anaerobic or microaerobic conditions, availability of NADH for the reduction of the precursors into 1,2-propanediol is advantageously increased. This is obtained by alleviating the repression on the tricarboxylic acid cycle mediated by the global regulator ArcA (encoded by the arcA gene). NADH concentration in the cell can also be increased by inactivating the NADH dehydrogenase II encoded by the gene ndh. Therefore, preferably, at least one gene selected among arcA and ndh is attenuated.

Especially under anaerobic or microaerobic conditions, it is advantageous that the pyruvate dehydrogenase complex (PDC), converting pyruvate into acetyl-coA has low sensitivity to inhibition by NADH. Lower sensitivity is defined with reference to the sensitivity of the wild-type enzyme. Such characteristic can be obtained by a specific mutation in the lpd gene (coding for the sub-unit lipoamide dehydrogenase of the PDC) resulting in the replacement of alanine 55 in the protein sequence of the enzyme by the residue valine.

Preferentially the microorganism designed to produce mainly 1,2-propanediol is selected among bacteria, yeasts or fungi. More preferentially, the microorganism is selected among Enterobacteriaceae, Bacillaceae, Clostridiaceae, Streptomycetaceae and Corynebacteriaceae. Even more preferentially, the microorganism is either Escherichia coli or Clostridium acetobutylicum.

Specifically for the production of acetol, it may be advantageous to increase the specific enzyme activities leading to the formation of this compound: methylglyoxal synthase and methylglyoxal reductase. Advantageously, the enzyme activities are increased by at least 50%, preferably by at least 100%, compared to the native activity of the corresponding non-modified microorganism.

To obtain the increase of the specific enzymatic activities, the genes coding for these activities are preferentially overexpressed: the mgsA gene, coding for methylglyoxal synthase, yqhD, yafB, ydhF, ycdW, yqhE, yeaE, yghZ, yajO, tas, ydjG, and ydbC, all coding for methylglyoxal reductases.

The combination of the overexpression of the mgsA and yqhD genes is preferentially used.

In addition, for the production of acetol, it is advantageous to prevent the formation of 1,2-propanediol from acetol. This result can be achieved by attenuating the activity of at least one enzyme involved in the conversion of acetol into 1,2-propanediol. Preferentially, the expression of the gldA gene is attenuated, more preferentially, the gldA gene is deleted.

Other genes whose expression may advantageously be attenuated are the following: pts G, ptsH, ptsI, crr, edd, eda, gloA, aldA, aldB, ldhA, pflA, pflB, adhE, tpiA, gapA, pykA, pykF, ackA, pta, poxB.

Other genes whose expression may advantageously be enhanced are the following: galP, glk, ppsA.

Preferentially, the microorganism designed to produce mainly acetol is selected among bacteria, yeast or fungi. More preferentially, the microorganism is selected among Enterobacteriaceae, Bacillaceae, Streptomycetaceae and Corynebacteriaceae. Even more preferentially, the microorganism is either Escherichia coli or Klebsiella pneumoniae.

It is also an object of the present invention to provide a microorganism with enhanced glyoxalase III activity, wherein the polynucleotide of the invention is overexpressed.

Preferentially, the overexpression is obtained by transforming the organism with the vector of the invention or by integrating the polynucleotide or the cassette of the invention into the chromosome of the organism.

One single copy or multiple copies of the gene borne by expression vectors or integrated into the chromosome can be introduced in order to modulate the overexpression. In addition, different kind of promoters inducing different level of expression of the gene can be used.

The adequate position on the chromosome for the insertion of the new gene can be selected by the expert in the art. This position (or locus) should not affect the essential functions of the host organism. Methods for integration of a gene into the chromosome of a host organism are disclosed for example in Sambrook et al (1989).

It is an object of the present invention to provide a microorganism with enhanced glyoxalase III activity, wherein expression of the native gene coding for the polypeptide of the invention is enhanced. This is preferentially obtained by introducing a strong promoter upstream the coding sequence of the native gene.

Additionally, other regulatory elements of the gene can be modified. For example, suitable mutations that can be selected by the expert in the field in the upstream region of the gene (start codon, ribosome binding site) can results in increased expression. In addition, an inducible promoter can be introduced in order to turn on/off the expression of the gene when desired.

In a preferred embodiment of the invention, a strong promoter is present upstream to the coding sequence of the native gene coding for the polypeptide according to the invention.

The present invention is also related to a microorganism with enhanced glyoxalase III activity, which is further modified to enhance production of lactate.

The term “lactate” as used herein designates D-lactate and L-lactate and mixtures thereof, including mixtures in different proportions such as 50/50 (racemic mixture), 75/25, 90/10 and 100/0.

In a specific embodiment of the invention, modifications are introduced into the microorganism such as described previously, to enhance specifically the production of lactate.

Preferentially, the methylglyoxal synthase activity is increased. The preferred method is the overexpression of the mgsA gene. Additionally, one or several mutation can be introduced in the mgsA gene in order to increase the methylglyoxal synthase activity under the culture conditions used.

Other genes whose expression may advantageously be enhanced are the following: galP, glk, ppsA.

In another embodiment of the invention, at least one of the enzyme activities of the Entner-Doudoroff pathway encoded by the genes edd and eda is attenuated. Preferentially, at least one of the genes edd or eda is attenuated. As already mentioned, the Entner-Doudoroff pathway can function as an unwanted bypass of the glycolysis pathway.

As for the production of 1,2-propanediol and/or acetol, the re-direction of the carbon flux toward the methylglyoxal bypass is advantageous. Therefore, the attenuation of the GAPDH and the features associated (engineering of sugar import or engineering of PEP recycling) are preferentially introduced.

Preferentially, in the microorganism according to the invention, some enzyme activities involved in by-product formation pathways are attenuated in order to increase the yield of lactate:

The pyruvate formate lyase activity, responsible for the synthesis of acetyl-CoA and formate from pyruvate, encoded by the genes pflA and pflB.

The fumarate reductase activity, responsible for the synthesis of succinate from fumarate, encoded by the operon frdABCD.

The alcool-aldehyde dehydrogenase activity, responsible for the synthesis of ethanol from acetyl-CoA, encoded by the gene adhE.

The phosphotransacetylase and acetate kinase activities, responsible for the synthesis of acetate in two steps from acetyl-CoA, encoded respectively by the genes pta and ackA.

The pyruvate oxidase activity, responsible for the synthesis of acetate in one step from pyruvate, encoded by the gene poxB.

Preferentially, the attenuation of activity is obtained by the attenuation of at least one of these genes.

Other potential by-products originating from the methylglyoxal bypass are acetol, lactaldehyde and 1,2-propanediol. In order to prevent the formation of these by-products, at least one methylglyoxal reductase activity is attenuated. This is preferentially realized by attenuating at least one gene coding for a methylglyoxal reductase activity chosen among: yqhD, yafB, yqhE, ydhF, ycdW, yeaE, yghZ, yajO, tas, ydjG, ydbC and gldA.

It is advantageous that the D-lactate produced is not further metabolized. Therefore, in another embodiment of the invention, at least one enzyme activity utilizing D-lactate is attenuated. Preferentially, the did gene is attenuated.

It is also advantageous to prevent a further metabolisation of L-lactate. Preferentially, the expression or activity at least one enzyme utilizing L-lactate is attenuated. More preferentially, the lldD gene is attenuated.

Other genes whose expression may advantageously be attenuated to favour the production of lactate are the following: pts G, ptsH, ptsI, crr, gloA, aldA, aldB, gapA, pykA, pykF, tpiA.

Preferentially the microorganism designed to produce lactate is selected among bacteria, yeasts or fungi. More preferentially, the microorganism is selected among Enterobacteriaceae, Bacillaceae, Streptomycetaceae and Corynebacteriaceae. Even more preferentially, the microorganism is either from the species Escherichia coli, Bacillus subtilis or Corynebacterium glutamicum.

The present invention provides for a method for modulating the glyoxalase III enzymatic activity in a microorganism, wherein activity of the polypeptide of the invention is enhanced or attenuated in said microorganism.

Preferentially, in said method, the glyoxalase III enzymatic activity is enhanced by overexpressing the polynucleotide of the invention.

Preferentially, in said method, the glyoxalase III enzymatic activity is attenuated by attenuating the expression of the polynucleotide of the invention.

The invention is also related to a method for preparing 1,2-propanediol and/or acetol, wherein a microorganism according to the invention is grown in an appropriate culture medium comprising a source of carbon, and the produced 1,2-propanediol and/or acetol is recovered. The production of 1,2-propanediol is performed under aerobic, microaerobic or anaerobic conditions. The production of acetol is performed under aerobic or microaerobic conditions, preferentially under aerobic conditions.

The term “carbon substrate” or “source of carbon” means any carbon source capable of being metabolized by a microorganism wherein the substrate contains at least one carbon atom. Authors refer particularly to renewable, inexpensive and fermentable carbon source such as monosaccharides, oligosaccharides, polysaccharides, single-carbon substrates, and polyols such as glycerol. Saccharides of the formula (CH₂O)ₙ are also called oses or “simple sugars”; monosaccharides include fructose, glucose, galactose and mannose. Other carbon sources are disaccharides, trisaccharides, oligosaccharides and polysaccharides. Disaccharides include saccharose (sucrose), lactose and maltose. Starch and hemicellulose are polysaccharides, also known as “complex sugars”.

Advantageously the recovered 1,2-propanediol and/or acetol is furthermore purified.

The invention is also related to a method for preparing lactate, wherein a microorganism according to the invention is grown in an appropriate growth medium containing a carbon source, and the lactate is recovered. The production of lactate is performed under aerobic, microaerobic or anaerobic conditions, preferentially under aerobic conditions.

Advantageously, the recovered lactate is furthermore purified.

The culture conditions for fermentation processes can be readily defined by those skilled in the art. In particular, bacteria are fermented at temperatures between 20° C. and 55° C., preferably between 25° C. and 40° C., and preferably at about 35° C. for C. acetobutylicum and at about 37° C. for E. coli and K. pneumoniae.

This process can be carried out either in a batch process, in a fed-batch process or in a continuous process.

“Under aerobic conditions” means that oxygen is provided to the culture by dissolving the gas into the liquid phase. This could be obtained by (1) sparging oxygen containing gas (e.g. air) into the liquid phase or (2) shaking the vessel containing the culture medium in order to transfer the oxygen contained in the head space into the liquid phase. Advantage of the fermentation under aerobic conditions instead of anaerobic conditions is that the presence of oxygen as an electron acceptor improves the capacity of the strain to produce more energy in form of ATP for cellular processes. Therefore the strain has its general metabolism improved.

Micro-aerobic conditions are defined as culture conditions wherein low percentages of oxygen (e.g. using a mixture of gas containing between 0.1 and 10% of oxygen, completed to 100% with nitrogen), is dissolved into the liquid phase.

Anaerobic conditions are defined as culture conditions wherein no oxygen is provided to the culture medium. Strictly anaerobic conditions are obtained by sparging an inert gas like nitrogen into the culture medium to remove traces of other gas. Nitrate can be used as an electron acceptor to improve ATP production by the strain and improve its metabolism.

The term “appropriate growth medium” according to the invention denotes a medium of known molecular composition adapted to the growth of the micro-organism. For example, a mineral culture medium of known set composition adapted to the bacteria used, containing at least one carbon source. In particular, the mineral growth medium for E. coli or K. pneumoniae can thus be of identical or similar composition to M9 medium (Anderson, 1946, Proc. Natl. Acad. Sci. USA 32:120-128), M63 medium (Miller, 1992; A Short Course in Bacterial Genetics: A Laboratory Manual and Handbook for Escherichia coli and Related Bacteria, Cold Spring Harbor Laboratory Press, Cold Spring Harbor, N.Y.) or a medium such as that defined by Schaefer et al. (1999, Anal. Biochem. 270: 88-96).

The carbon source used for the culture of E. coli or K. pneumoniae is preferentially a simple carbon source and can be arabinose, fructose, galactose, glucose, lactose, maltose sucrose or xylose. An especially preferred simple carbon source is glucose.

The invention is described above, below and in the Examples with respect to E. coli. Thus the genes that can be attenuated, deleted or over-expressed for the initial and evolved strains according to the invention are defined mainly using the denomination of the genes from E. coli. However, this designation has a more general meaning according to the invention, and covers the corresponding genes in other micro-organisms. Using the GenBank references of the genes from E. coli, those skilled in the art can determine equivalent genes in other organisms than E. coli.

The means of identification of the homologous sequences and their percentage homologies are well-known to those skilled in the art, and include in particular the BLAST programmes that can be used on the website http://www.ncbi.nlm.nih.gov/BLAST/ with the default parameters indicated on that website. The sequences obtained can be exploited (aligned) using for example the programmes CLUSTALW (http://www.ebi.ac.uk/clustalw/), with the default parameters indicated on these websites.

The PFAM database (protein families database of alignments and hidden Markov models http://www.sanger.ac.uk/Software/Pfam/) is a large collection of alignments of protein sequences. Each PFAM makes it possible to visualise multiple alignments, view protein domains, evaluate distributions among organisms, gain access to other databases and visualise known protein structures.

COGs (clusters of orthologous groups of proteins http://www.ncbi.nlm.nih.gov/COG/) are obtained by comparing protein sequences derived from 66 fully sequenced unicellular genomes representing 14 major phylogenetic lines. Each COG is defined from at least three lines, making it possible to identify ancient conserved domains.

### REFERENCES IN THE ORDER OF THE CITATION IN THE TEXT

1. Bunch P K, Mat-Jan F, Lee N, Clark D P (1997), Microbiology 143: 187-195
2. Garvie E I (1980), Microbiol. Rev. 44: 106-139
3. Cooper R A (1984), Annu. Rev. Microbiol. 38: 49-68 Arch. Microbiol. 170: 209-219
4. Rule G S, Pratt E A, Chin C C Q, Wold F, Ho C (1985), J. Bacteriol. 161: 1059-1068
5. Dong J M, Taylor J S, Latour D J, Iuchi S, Lin E C C (1993), J. Bacteriol. 175: 6671-6678
6. Ferguson G P, Toetmeyer S, MacLean M J, Booth I R (1998), Arch. Microbiol. 170: 209-219
7. Grabar T B, Zhou S, Shanmugam K T, Yomano L P, Ingram L O (2006), Biotechnol. Lett. 28: 1527-1535
8. Misra K, Banerjee A R, Ray S, Ray M (1995), Biochem. J. 305: 999-1003
9. MacLean M J, Ness L S, Ferguson G P, Booth I R (1998), Mol. Microbiol. 27: 563-571
10. Okado-Matsumoto A and Fridovich I (2000), J. Biol. Chem. 275: 34853-43857
11. Benov L, Sequeira F, Beema A (2004), Acta Biochimica Polonica 51: 857-860
12. Tran Din K and Gottschalk G (1985), Arch. Microbiol. 142: 87-92
13. Cameron D C and Cooney C L (1986), Bio/Technology, 4: 651-654
14. Sanchez-Rivera F, Cameron D C, Cooney C L (1987), Biotechnol. Lett. 9: 449-454
15. Cameron D C, Altaras N E, Hoffman M L, Shaw A J (1998), Biotechnol. Prog. 14: 116-125
16. Altaras N E and Cameron D C (1999), Appl. Environ. Microbiol. 65: 1180-1185
17. Altaras N E and Cameron D C (2000), Biotechnol. Prog. 16: 940-946
18. Huang K, Rudolph F B, Bennett G N (1999), Appl. Environ. Microbiol. 65: 3244-3247
19. Berrios-Rivera S J, San K Y, Bennett G N (2003), J. Ind. Microbiol. Biotechnol. 30: 34-40
20. Chang D E, Jung H C, Rhee J S, Pan J G (1999), Appl. Environ. Microbiol. 65: 1384-1389
21. Zhou S, Causey T B, Hasona A, Shanmugam K T, Ingram L O (2003a), Appl. Environ. Microbiol. 69: 399-407
22. Dien B S, Nichols N N, Bothast (2001), J. Ind. Microbiol. Biotechnol. 27: 259-264
23. Zhou S, Shanmugam K T, Ingram L O (2003b), Appl. Environ. Microbiol. 69: 2237-2244
24. Sastry M S R, Korotkov K, Brodsky J, Baneyx F (2002), J. Biol. Chem. 277: 46026-46034
25. Lee S J, Kim S J, Kim I K, Ko J, Jeong C S, Kim G H, Park C, Kang, S O, Suh, P G, Lee H S, Cha S S (2003), Biol. Chem. 278: 44552-44559
26. Quigley P M, Korotkov K, Baneyx F, Hol W G J (2003), Proc. Natl. Acad. Sci. USA 100: 3137-3142
27. Zhao Y, Liu D, Kaluarachchi W D, Bellamy H D, White M A, Fox R O (2003) Protein Science 12: 2303-2311
28. Malki A, Kern R, Abdallah J, Richarme G (2003), Biochem. Biophys. Res. Com. 301: 430-436
29. Malki A, Caladas T, Kern R, Abdallah J, Eckey V, Kim S J, Cha S S, Mori H, Richarme G (2005), J. Biol. Chem. 280: 14420-14426
30. Sambrook J, Russell D W (2001) Molecular Cloning: A Laboratory Manual. Cold Spring Harbor Laboratory Press, Cold Spring Harbor, N.Y.
31. Datsenko K A and Wanner B L (2000), Proc. Natl. Acad. Sci. USA 97: 6640-6645
32. Anderson E H (1946), Proc. Natl. Acad. Sci. USA 32:120-128
33. Miller (1992), A Short Course in Bacterial Genetics: A Laboratory Manual and Handbook for Escherichia coli and Related Bacteria, Cold Spring Harbor Laboratory Press, Cold Spring Harbor, N.Y.
34. Schaefer U, Boos W, Takors R, Weuster-Botz D (1999), Anal. Biochem. 270: 88-96
35. Bradford M M (1976), Anal. Biochem. 72: 248-264

### DRAWING

FIG. 1: SDS 4-15%—Gradient polyacrylamide gel

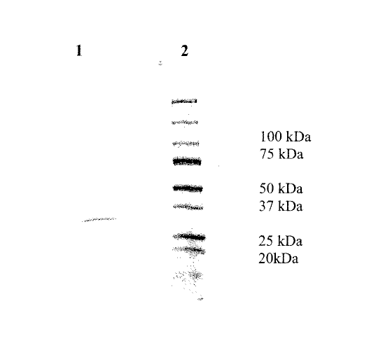

Lane 1: Fraction of Gel filtration column containing the glyoxalase activity,

Lane 2: Molecular weight marker

### EXAMPLES

### Example 1

### Purification of Glyoxalase III Activity in E. Coli PG0016 and Identification of the Encoding Gene

1. PG0016 Strain Construction

1.1. Construction of a Modified Strain E. Coli MG1655 lpd*, ΔtpiA, ΔpflAB, ΔadhE, ldhA::km, ΔgloA, ΔaldA, ΔaldB, Δedd.

The chloramphenicol resistance cassette was eliminated in the strain E. coli MG1655 lpd*, ΔtpiA, ΔpflAB, ΔadhE, ldhA::Km, ΔgloA, ΔaldA, ΔaldB, Δedd::Cm (See WO2005073364) according to Protocol 1.

Protocol 1: Elimination of Resistance Cassettes (FRT System)

The chloramphenicol and/or kanamycin resistance cassettes were eliminated according to the following technique. The plasmid pCP20 carrying the FLP recombinase acting at the FRT sites of the chloramphenicol and/or kanamycin resistance cassettes was introduced into the strain by electroporation. After serial culture at 42° C., the loss of the antibiotic resistance cassettes was checked by PCR analysis with the oligonucleotides given in Table 1.

The presence of the modifications previously built in the strain was checked using the oligonucleotides given in Table 1.

The strain obtained was named E. coli MG1655 lpd*, ΔtpiA, ΔpflAB, ΔadhE, ldhA::Km, ΔgloA, ΔaldA, ΔaldB, Δedd.

TABLE 1
Oligonucleotides used for checking the insertion of a
resistance cassette or the loss of a resistance cassette

| | | | Homology with |
|---|---|---|---|
| Region name | Names of oligos | SEQ ID | chromosomal region |
| tpiA gene | Cdh | N^{o}3 | See WO2005073364 |
| (deletion) | YIIQ | N^{o}4 | |
| pflAB gene | pflABF | N^{o}5 | See WO2005073364 |
| | pflABR | N^{o}6 | |
| adhE gene | ychGf | N^{o}7 | See WO2005073364 |
| | adhECr | N^{o}8 | |
| ldhA gene | hsIJC | N^{o}9 | See WO2005073364 |
| (cassette insertion) | ldhAC2 | N^{o}10 | |
| gloA gene | NemACd | N^{o}11 | See WO2005073364 |
| | Rnt Cr | N^{o}12 | |
| aldA gene | Ydc F C f | N^{o}13 | See WO2005073364 |
| | gapCCr | N^{o}14 | |
| aldB gene | aldB C f | N^{o}15 | See WO2005073364 |
| | YiaYCr | N^{o}16 | |
| edd gene | Eda d | N^{o}17 | See WO2005073364 |
| | Zwf r | N^{o}18 | |
| ldhA gene | ldhAF | N^{o}19 | 1439724 to 1439743 |
| (deletion) | ldhAR | N^{o}20 | 1441029 to 1441007 |
| yedU gene | yedUF | N^{o}21 | 2033421 to 2033441 |
| | yedUR | N^{o}22 | 2035349 to 2035328 |
| yqhD gene | yqhDF | N^{o}23 | See WO 2008/115863 |
| | yqhDR | N^{o}24 | |

1.2. Construction of a Modified Strain E. Coli MG1655 lpd*, ΔtpiA, ΔpflAB, ΔadhE, ΔldhA::cm, ΔgloA, ΔaldA, ΔaldB, Δedd.

In order to eliminate the kanamycin resistance cassette and to inactivate the ldhA gene, the chloramphenicol resistance cassette was inserted into the ldhA gene deleting most of the gene concerned according to Protocol 2.

Protocol 2: Introduction of a PCR Product for Recombination and Selection of the Recombinants (FRT System).

The oligonucleotides chosen and given in Table 2 for replacement of a gene or an intergenic region were used to amplify either the chloramphenicol resistance cassette from the plasmid pKD3 or the kanamycin resistance cassette from the plasmid pKD4 (Datsenko, K. A. & Wanner, B. L. (2000)). The PCR product obtained was then introduced by electroporation into the recipient strain bearing the plasmid pKD46 in which the system λ Red (γ, β, exo) expressed greatly favours homologous recombination. The antibiotic-resistant transformants were then selected and the insertion of the resistance cassette was checked by PCR analysis with the appropriate oligonucleotides given in Table 1.

The other modifications of the strain were checked with the oligonucleotides given in Table 1.

The resulting strain was named E. coli MG1655 lpd*, ΔtpiA, ΔpflAB, ΔadhE, ΔgloA, ΔaldA, ΔaldB, Δedd or PG0016.

TABLE 2
Oligonucleotides used for replacement of a
chromosomal region by recombination with a PCR product

| | | | Homology with |
|---|---|---|---|
| Region name | Names of oligos | SEQ ID | chromosomal region |
| ldhA gene | DldhAF | N^{o}25 | 1440865-1440786 |
| | DldhAR | N^{o}26 | 1439878-1439958 |
| yedU gene | DyedUF | N^{o}27 | 2033857-2033938 |
| | DyedUR | N^{o}28 | 2034708-2034628 |

2—Glyoxalase III Activity Assay

The glyoxalase III enzyme activity was determined in vitro in E. coli cell-free extracts. Biomass harvested by centrifugation was resuspended in 100 mM Potassium Phosphate buffer pH 7.6, 10% sucrose, 1 mM DTT, 0.1 mM PLP, 1 mM EDTA, and a protease inhibitor cocktail (Roche) and sonicated on ice (Branson sonifier, 70 W) during four cycles of 30 sec with 30 sec intervals. After centrifugation, the supernatant corresponding to the crude extract was desalted using an Econo-Pac 10 DG column (BioRad). Protein concentration in the desalted supernatant was measured by a Bradford colorimetric assay (Bradford, 1976).

One hundred μL of desalted extract were incubated during either 5 or 30 minutes at 37° C. in a reaction mix containing 50 mM Potassium phosphate pH 8 and 5 mM methylglyoxal in a total volume of 250 μL. After the incubation time, 1 ml of −20° C. acetone was then added as well as 100 μL of the internal standard L-Serine[1-13 C] at a concentration of 1.5 M in a total volume of 1.5 mL. The reaction mix were incubated at −20° C. for 30 min and centrifugated for 5 min at 10000 g. The supernatants were frozen at −80° C. and lyophilized overnight. The following day, the dried samples were silylated by the addition of 0.5 ml hydroxylamine 20% (diluted in pyridine) and incubated for 1 h30 at 30° C. followed by the addition of 0.5 ml tert-butyldimethylsilyltrifluoroacetamide (TBDMSTFA) and 0.5 ml of pyridine and incubation for 1 hour at 60° C. The samples were analyzed by GC-MS (Agilent GC6890-MS5973, column Varian DB5MS) and the quantity of lactate produced by the Glyoxalase III enzyme after 5 min and 30 min was measured by using a standard curve of lactate (0 to 33 μM). The total activity (nmoles/min) in the crude extract was calculated using the quantity of lactate produced between 5 and 30 min. The protein concentration was used to determine the specific activity in nmoles/min/mg (mUI/mg).

3—Purification of Glyoxalase III Activity in PG0016 Strain

The strain PG0016 was cultivated in a 21 batch fermenter with a 1.4 l working volume. The culture medium was based on a minimal medium with 10 g/l glucose supplemented with yeast extract. The temperature of the culture was maintained constant at 37° C. and the pH was permanently adjusted to 6.8 using an NH₄OH solution. The agitation rate was adjusted according to the oxygen demand. The concentration of dissolved oxygen is maintained at values between 30 and 40% saturation by using a gas controller. When the optical density reached a value of 2, the culture was stopped and the biomass was recovered by centrifugation.

All chromatographic columns were run at room temperature. Fractions were stored at −80° C. between purification steps.

Step 1: Preparation of Cell-Free Extracts

320 mg of PG0016 E. coli biomass were resuspended in 48 ml of 100 mM Potassium Phosphate buffer pH 7.6, 10% sucrose, 1 mM DTT, 0.1 mM PLP, 1 mM EDTA, and a protease inhibitor cocktail. Cells were sonicated on ice (Branson sonifier, 70 W) during four cycles of 30 sec with 30 sec intervals. The suspension was treated with DNase I (100 U/ml) and 1 mM MgCl₂ for 30 min at room temperature under stirring. Cell debris were removed by centrifugation at 12000 g for 30 min. The crude extract was desalted using an Econo-Pac 10 DG column (BioRad).

The glyoxalase III specific activity of in this desalted extract was 33 mUI/mg.

Step 2: Ammonium Sulphate Precipitation

The crude extract was precipitated at a concentration of 40% ammonium sulphate: solid ammonium sulphate (226 g/l) was added to the crude extract while stirring. After 15 min of incubation at room temperature, the sample was centrifugated at 12000 g for 30 min at 4° C. and the pellet was discarded. The glyoxalase III specific activity in this supernatant was 102 mUI/mg.

Step 3: Hydrophobic Chromatography

Using an Akta Purifier (GE Healthcare), the 40% ammonium sulphate supernatant was loaded onto a 5 ml HiTrap PhenylHP column (GE Healthcare) equilibrated with 20 mM Tris buffer pH 7.5, 10% sucrose, 1 mM DTT, 1.2 M ammonium sulphate. The column was then washed with 10 column volumes of the same buffer. Proteins were eluted with a linear decreasing gradient of 20 column volumes from 1.2 M to 0 M ammonium sulphate. After elution, the column was washed with 10 column volumes of 20 mM Tris buffer pH 7.5, 10% sucrose. The flow rate of the column was 2.5 ml/min and 2.5 ml fractions were collected.

Fractions from the HiTrap PhenylHP column were assayed for glyoxalase III activity. The protein was eluted between 0.4 and 0.2 M ammonium sulphate. The glyoxalase III specific activity of the most active fractions was between 150 and 540 mUI/mg. The most active fractions were pooled and dialysed against 20 mM Tris pH 8, 1 mM DTT, 10% sucrose.

Step 4: Anionic Chromatography pH 8

The dialysed pool was applied to a 1 ml Resource Q column (GE Healthcare) equilibrated with 20 mM Tris buffer pH8, 1 mM DTT, 10% sucrose. The column was then washed with 10 column volumes of the same buffer. Proteins were eluted with a linear gradient from 0 M to 0.5 NaCl of 20 column volumes. The column was washed with 10 column volumes of 20 mM Tris pH8, 1 mM DTT, 10% sucrose 1M NaCl. The flow rate of the column was 1 ml/min and 0.5 ml fractions were collected. Fractions from Resource Q column were assayed for glyoxalase III activity. The protein was eluted with 190 mM NaCl. The most active fraction was concentrated for gel filtration. The glyoxalase III specific activity of this fraction was 1594 mUI/mg.

Step 5: Gel Filtration

The concentrated fraction from the Resource Q column was loaded onto a Superdex 200 10/300 GL column (GE Healthcare) equilibrated with 50 mM potassium phosphate buffer pH 7, 150 mM NaCl. The flow rate of the column was 0.5 ml/min and 0.5 ml fractions were collected. Fractions were assayed for glyoxalase activity. The protein was eluted with 14.5 ml of buffer. According to the calibration of the column, this elution volume corresponds to a protein of about 60 kDa.

The most active fraction with a glyoxalase III specific activity of 4718 mUI/mg was applied to and run out on a SDS 4-15% gradient polyacrylamide gel. This gel (Fig) showed a major band at about 30 kDa.

TABLE 3
summary of purification of glyoxalase III activity

| | Specific | | Total |
|---|---|---|---|
| | activity | Purification | purification |
| Purification steps | (mUI/mg) | fold | fold |
| Desalted Extract | 33 | 1 | 1 |
| Ammonium sulphate | 102 | 3.1 | 3.1 |
| precipitation (40%) | | | |
| HiTrap Phenyl HP | 150 (F18) | 1.5 | 4.5 |
| column fractions | 538 (F19) | 5.3 | 16.3 |
| | 397 (F20) | 3.9 | 12.0 |
| Resource Q | 1594 | 15.3 | 48.3 |
| Superdex 200 | 4718 | 3.0 | 143 |

4—Identification of the Gene Coding for Glyoxalase III Activity

The region of the gel corresponding to the protein at 30 kDa was cut off using a sterile pipette tip. This gel plug was then used for identification of proteins by mass spectroscopy.

The sample was subjected to trypsin digestion and analyzed by nano LC/MS/MS on a CapLC-Q-TOF2 (Waters) and by MALDI on MALDI MX (Waters). The candidate proteins were identified with the softwares ProteinLynx Global Server (Waters) and Mascot (Matrix Science) using the protein data bank of E. coli. For both analyses, there was only one protein identified with a significant score, this protein was the protein HchA of 31 kDa also called Hsp31 (accession P31658). The corresponding gene was called hchA or yedU.

### Example 2

### Modulation of Glyoxalase III Activity in E. Coli

1—Disruption of yedU Gene in E. Coli and Validation of Loss of Glyoxalase III Activity

1.1. Construction of the Strain PG0016 without Antibiotic Resistance Cassette

The chloramphenicol resistance cassette was eliminated in the strain E. coli MG1655 lpd*, ΔtpiA, ΔpflAB, ΔadhE, ΔldhA::Cm, ΔgloA, ΔaldA, ΔaldB, Δedd (PG0016) according to Protocol 1.

The resulting strain E. coli MG1655 lpd*, ΔtpiA, ΔpflAB, ΔadhE, ΔldhA, ΔgloA, ΔaldA, ΔaldB, Δedd was named PG0021.

1.2. Construction of a Modified Strain E. Coli MG1655 ΔyedU::Cm

The gene yedU was inactivated in strain E. coli MG1655 by inserting a chloramphenicol antibiotic resistance cassette and deleting most of the gene concerned using the technique described in Protocol 2 with the oligonucleotides given in Table 2. The resulting strain was named E. coli MG1655 ΔyedU::Cm.

1.3. Construction of the Modified Strain PG0021 ΔyedU::cm

The deletion of the gene yedU by replacement of the gene by a chloramphenicol resistance cassette in the strain E. coli MG1655 lpd*, ΔtpiA, ΔpflAB, ΔadhE, ΔldhA, ΔgloA, ΔaldA, ΔaldB, Δedd (PG0021) was performed by the technique of transduction with phage P1.

Protocol 3: Transduction with Phage P1 for Deletion of a Gene

The deletion of the chosen gene by replacement of the gene by a resistance cassette (kanamycin or chloramphenicol) in the recipient E. coli strain was performed by the technique of transduction with phage P1. The protocol was in two steps, (1) the preparation of the phage lysate on the strain MG1655 with a single gene deleted and (ii) the transduction of the recipient strain by this phage lysate.

Preparation of the Phage Lysate
Seeding with 100 μl of an overnight culture of the strain MG1655 with a single gene deleted of 10 ml of LB+Cm 30 μg/ml+glucose 0.2%+CaCl₂ 5 mM.
Incubation for 30 min at 37° C. with shaking.
Addition of 100 μl of phage lysate P1 prepared on the wild type strain MG1655 (approx. 1×10⁹ phage/ml).
Shaking at 37° C. for 3 hours until all cells were lysed.
Addition of 200 μl of chloroform, and vortexing.
Centrifugation for 10 min at 4500 g to eliminate cell debris.
Transfer of supernatant in a sterile tube and addition of 200 μl of chloroform.
Storage of the lysate at 4° C.

Transduction
Centrifugation for 10 min at 1500 g of 5 ml of an overnight culture of the E. coli recipient strain in LB medium.
Suspension of the cell pellet in 2.5 ml of MgSO₄ 10 mM, CaCl₂ 5 mM.
Control tubes: 100 μl cells
   100 μl phages P1 of the strain MG1655 with a single gene deleted.
Tube test: 100 μl of cells+100 μl phages P1 of strain MG1655 with a single gene deleted.
Incubation for 30 min at 30° C. without shaking.
Addition of 100 μl sodium citrate 1 M in each tube, and vortexing.
Addition of 1 ml of LB.
Incubation for 1 hour at 37° C. with shaking
Plating on dishes LB+Cm 30 μg/ml after centrifugation of tubes for 3 min at 7000 rpm.
Incubation at 37° C. overnight.

The antibiotic-resistant transformants were then selected and the insertion of the deletion was checked by a PCR analysis with the appropriate oligonucleotides given in Table 1.

The other modifications of the strain were checked with the oligonucleotides given in Table 1.

The resulting strain was named E. coli MG1655 lpd*, ΔldhA, ΔtpiA, ΔpflAB, ΔadhE, ΔgloA, ΔaldA, ΔaldB, Δedd, ΔyedU::cm (PG0021 ΔyedU::cm)

1.4. Production of Biomass of the Strain PG0021 ΔyedU::Cm and Activity Assay

The strain PG0021 ΔyedU::Cm and the control strain PG0021 were cultivated at 37° C. under aerobic conditions in 500 ml baffled Erlenmeyer flasks in minimal medium with 10 g/l glucose and buffered with MOPS. The pH was adjusted at 6.8 at the beginning of the cultures. The flasks were agitated at 200 rpm on an orbital shaker. The biomass was harvested by centrifugation when the cultures reached an optical density measured at 550 nm above 5 units. Cell-free extracts were prepared and glyoxalase III activity assays were carried out as described previously.

The glyoxalase III activity of the parent strain PG0021 was 24 mUI/mg whereas the glyoxalase III activity of the strain PG0021 ΔyedU was 4 mUI/mg. The deletion of the yedU gene almost abolished the glyoxalase III activity of strain PG0021.

2—Overexpression of yedU Gene in E. Coli and Validation of Glyoxalase III Activity Increase

2.1. Construction of Plasmid pME101

For the expression of the yedU gene under the control of the trc promoter from a low copy vector the plasmid pME101 was constructed as follows. The plasmid pCL1920 (Lerner & Inouye, 1990, NAR 18, 15 p 4631—GenBank AX085428) was PCR amplified using the oligonucleotides PME101F and PME101R and the BstZ171-XmnI fragment from the vector pTrc99A (Amersham Pharmacia Biotech, Piscataway, N.J.) harboring the lacI gene and the trc promoter was inserted into the amplified vector.

PME101F (SEQ ID N^{o} 29):
ccgacagtaagacgggtaagcctg
PME101R (SEQ ID N^{o} 30):
agcttagtaaagccctcgctag

2.2. Construction of Plasmid pME101-yedU

The gene yedU was PCR amplified from genomic DNA of E. coli MG1655 using the following oligonucleotides:

yedUF2, consisting of 34 bases (SEQ ID No 31):

catgtcatgactgttcaaacaagtaaaaatccgc

with:

a region (underlined letters) homologous to the sequence (2033857-2033884) of the gene yedU, and

a restriction site BspHI (bold face letters)

yedUR2, consisting of 28 bases (SEQ ID No32):

CTAcccgggCATAGGGCTTCAGTACGCC

with:

a region (underlined letters) homologous to the sequence (2034815-2034797) of the gene yedU, and

a restriction site SmaI (bold face letters).

The PCR amplified fragment was cut with the restriction enzymes BspHI and SmaI and cloned into the NcoI/SmaI sites of the vector pME101. The resulting plasmid was named pME101-yedU.

The plasmid pME101-yedU was introduced by electroporation into the strain E. coli MG1655. The strain obtained was named E. coli MG1655 (pME101-yedU).

2.3. Production of Biomass of the Strain E. Coli MG1655 pME101-yedU and Activity Assay

The E. coli strain MG1655 (pME101-yedU) and the control strain E. coli MG1655 were cultivated at 37° C. under aerobic conditions in 500 ml baffled Erlenmeyer flasks in minimal medium with 10 g/l glucose and buffered with MOPS. The pH was adjusted at 6.8 and 100 μM IPTG was added at the beginning of the cultures. The flasks were agitated at 200 rpm on an orbital shaker. The biomass was harvested by centrifugation when the cultures reached an optical density measured at 550 nm above 7 units. Cell-free extracts were prepared and glyoxalase III activity assays were carried out as described previously.

The glyoxalase III activity of the MG1655 strain was 8 mUI/mg whereas the glyoxalase III activity of the MG1655 strain overexpressing yedU was 142 mUI/mg. The glyoxalase III activity was increased 18 fold by overexpression of the yedU gene.

3—Determination of the Isomer of Lactate Produced by YedU

3.1. Purification of YedU Protein

The plasmid pETTOPO-yedU was built by amplifying the gene yedU from genomic DNA of E. coli MG1655 using the following oligonucleotides:

yedU pET F consisting of 29 bases (SEQ ID No 33)

cacc

[symbol]

[symbol]

[symbol]

with:

a region (italic bold letters) homologous to the sequence (2033859-2033883) of the gene yedU, and

a region (underlined letters) for the oriented cloning in pETTOPO

yedU pET R consisting of 21 bases (SEQ ID No 34)

( [symbol] acccgcgtaagctgccag)

with:

a region (italic bold letters) homologous to the sequence (2034690-2034692) of the gene yedU and corresponding to stop codon,

a region (underlined letters) homologous to the sequence (2034693-2034710) of the downstream region of the gene yedU

The PCR amplified fragment was cloned into the commercial vector pETTOPO (Invitrogen). The resulting plasmid was named pETTOPO-yedU. The plasmid pETTOPO-yedU was introduced by electroporation into the commercial strain E. coli BL21 star (Invitrogen), optimized for protein overexpression. The strain obtained was named E. coli BL21 star pETTOPO-yedU.

The E. coli strain BL21 star PETTOPO-yedU and the control strain E. coli BL21 star were cultivated at 37° C. under aerobic conditions in 500 ml baffled Erlenmeyer flasks with 50 ml LB medium with 2.5 g/l glucose. The flasks were agitated at 200 rpm on an orbital shaker. The temperature was decreased at 25° C. when the optical density (OD, measured at 550 nm) of the culture reached 0.8 OD units. The cultures were induced with 500 μM IPTG when the OD reached 2.4 units. The biomass was harvested by centrifugation when the cultures reached an OD above 3.5 units. Cell-free extracts were prepared and glyoxalase III activity assays were carried out as described previously. The glyoxalase III activity of the BL21 star control strain cannot be determined (below quantification level) whereas the glyoxalase III activity of the strain BL21 star PETTOPO-yedU was 192 mUI/mg. From this crude extract, the YedU was purified according to the previously described protocol (see Example 1). 180 μg of YedU protein were obtained.

35 μg of purified YedU protein were used to perform a Glyoxalase III activity assay as previously described. The isomers of lactate formed after reaction of the substrate methylglyoxal with the enzyme YedU were identified and quantified. Two analytical methods were used, either an enzymatic-spectrophotometric kit (Enzytec™ fluid D and L-lactic acid) based on the oxidation of D-lactate to pyruvate by NAD+ in the presence of D-lactate dehydrogenase (D-LDH) or L-lactate dehydrogenase (L-LDH), or an HPLC method using a chiral column (Chirex (D)-Penicillamine 150×4.6 mm, guard column Chirex (D)-Penicillamine 30×4.6 mm), which allows the separation of D and L-lactate. The results are presented in the table 4.

TABLE 4
Determination of the reaction product of methylglyoxal
with the purified YedU.

| | Purified hsp31 | | | | |
|---|---|---|---|---|---|
| | GC | | | Chiral column | |
| Sample | MS^{a} | Enzymatic kit^{b} | | (HPLC)^{c} | |
| Determination | Lactate | L-Lactate | D-Lactate | L-Lactate | D-Lactate |
| μM lactate | 794 | 336 | 325 | 789 | 602 |

| | | | |
|---|---|---|---|
| μM lactate | 794 | 661 | 1391 |
| total | | | |
| Ratio | | 0.97 | 0.76 |
| D-lactate/L- | | | |
| lactate | | | |

^{a}Quantification of lactate by GCMS
^{b}Quantification of D and L-lactate with the Enzymatic kit
^{c}Quantification of D and L-lactate with a chiral column by HPLC

The quantification of lactate using the GC MS or the enzymatic kit was similar. With the chiral column the quantity of lactate measured was higher than with the two others techniques. This difference can be explained by the quantification which was done using only one concentration of D and L lactate and not a range of concentration of D and L-lactate.

Regardless of the method used, it appears that YedU was able to convert the methyglyoxal into a mixture of D and L-lactate close to a racemic mixture.

### Example 3

### Improved Production of 1,2-Propanediol and Acetol with an E. Coli Strain with a Deletion of the yedU Gene

1—Construction of a Modified Strain E. Coli MG1655 lpd* ΔtpiA ΔpflAB ΔadhE ΔldhA ΔgloA ΔaldA ΔaldB Δedd ΔyedU::Cm

The construction of this strain was already described in Example 2.

2—Production of 1,2-Propanediol and Acetol with the Strain with a Deletion in the yedU Gene and with the Same Strain without Deletion

The strains built previously with a deletion in the yedU gene (ΔyedU strain) and without a deletion (Control strain) were cultivated for 25 hours under microaerobic conditions (70 ml closed Erlenmeyer flask filled with 21 ml of medium) in the medium given below with glucose as carbon source. The flasks were agitated at 200 rpm on an orbital shaker.

TABLE 5
composition of medium PG01_MC_V01 (pH adjusted to 6.5)

| Nutrient | Concentration (g/l) |
|---|---|
| CaCl₂2H₂O | 0.01 |
| CoCl₂6H₂O | 0.001 |
| MnSO₄H₂O | 0.03 |
| CuSO₄5H₂O | 0.0001 |
| H₃BO₃ | 0.0001 |
| Na₂MoO₄2H₂O | 0.00008 |
| ZnSO₄7H₂O | 0.001 |
| FeSO₄7H₂O | 0.05 |
| Citric acid | 0.0437 |
| Nitrilo Tri Acétic acid | 0.3 |
| (NTA) | |
| K₂HPO₄3H₂O | 1.4 |
| (NH₄)₂SO₄ | 1 |
| MgSO₄ | 0.2 |
| NaCl | 0.2 |
| NaHCO₃ | 0.2 |
| NaNO₃ | 0.42 |
| Yeast Extract | 4 |
| Thiamine HCl | 0.01 |
| Glucose | 10 |
| MOPS buffer | 41.8 |

The culture was carried out at 37° C. and the pH was maintained by buffering the culture medium with MOPS. At the end of the culture, 1,2-propanediol, acetol, lactate and residual glucose in the fermentation broth were analysed by HPLC and the yields of 1,2-propanediol+acetol and lactate over glucose were calculated. The results are given in the table below.

TABLE 6
Comparison of the production of 1,2-propanediol and acetol with
a strain deleted for the yedU gene and with the same strain without deletion

| | 1,2-propanediol + | | 1,2-propanediol + | |
|---|---|---|---|---|
| | Acetol | Lactate | Acetol yield | Lactate yield |
| Strain | titer (g/l) | titer (g/l) | (g/g glucose) | (g/g glucose) |
| ΔyedU strain | 1.15 +/− 0.02 | 0.02 +/− 0.02 | 0.41 +/− 0.01 | 0.01 +/− 0.01 |
| | (n = 3) | (n = 3) | (n = 3) | (n = 3) |
| Control strain | 1.29 +/− 0.03 | 0.20 +/− 0.01 | 0.35 +/− 0.02 | 0.05 +/− 0.01 |
| | (n = 3) | (n = 3) | (n = 3) | (n = 3) |

n is the number of repetitions of the same experiment.
The figures given are the mean and standard deviation of the figures obtained for n repetitions.

### Example 4

### Production of Lactate Under Microaerobic Conditions in an E. Coli Strain Overexpressing the yedU Gene

1—Construction of a Modified Strain E. Coli MG1655 lpd* ΔtpiA ΔpflAB ΔadhE ΔldhA ΔgloA ΔaldA ΔaldB Δedd ΔyqhD::Km pME101-VB01-yedU or pME101-yedU

The construction of the strain E. coli MG1655 lpd* ΔtpiA ΔpflAB ΔadhE ΔldhA ΔgloA ΔaldA ΔaldB Δedd ΔyqhD::Km was already described in patent application WO 2008/116853.

The plasmid pME101-VB01 was built according to the description given in patent application WO 2008/116848.

The gene yedU was PCR amplified from genomic DNA of E. coli MG1655 using the following oligonucleotides:

yedUF3, consisting of 34 bases (SEQ ID No 35):

catgtcatgactgttcaaacaagtaaaaatccgc

with:

a region (underlined letters) homologous to the sequence (2033857-2033884) of the gene yedU, and

a restriction site BspHI (bold face letters)

yedUR3, consisting of 24 bases (SEQ ID NO 36):

cattcaaacgtaatacgtattaac

with:

a region (underlined letters) homologous to the sequence (2034727-2034715 and 2034708-2034704) of the gene yedU, and

a restriction site SnaBI (bold face letters)

The PCR amplified fragment was cut with the restriction enzymes BspHI and SnaBI and cloned into the NcoI/SnaBI sites of the vector pME101-VB01. The resulting plasmid was named pME101-VB01-yedU.

The construction of the plasmid pME101-yedU was already described in Example 2.

The plasmids pME101-VB01-yedU or pME101-yedU were introduced into the modified strain E. coli MG1655 lpd* ΔtpiA ΔpflAB ΔadhE ΔldhA ΔgloA ΔaldA ΔaldB Δedd ΔyqhD::Km.

The strain obtained were named respectively E. coli MG1655 lpd* ΔtpiA ΔpflAB ΔadhE ΔldhA ΔgloA ΔaldA ΔaldB Δedd ΔyqhD::Km pME101-VB01-yedU (Strain 1) or E. coli MG1655 lpd* ΔtpiA ΔpflAB ΔadhE ΔldhA ΔgloA ΔaldA ΔaldB Δedd ΔyqhD::Km pME101-yedU (Strain 2).

2—Production of Lactate with the Strain with Overexpression of the yedU Gene and with the Strain without Overexpression

The strains built previously with overexpression of the yedU gene (Strain 1 and Strain 2) and without overexpression (Control strain) were cultivated for 46 h under microaerobic conditions (70 ml closed Erlenmeyer flask filled with 21 ml of medium) in the medium PG01_MC_V01 (see Example 3) with glucose as carbon source. The flasks were agitated at 200 rpm on an orbital shaker. The culture was carried out at 37° C. and the pH was maintained by buffering the culture medium with MOPS. The cultures of strain 1 and strain 2 were induced with 100 μM IPTG at the beginning of the culture in order to induce the expression of the yedU gene. At the end of the culture, lactate and residual glucose in the fermentation broth were analysed by HPLC and the yields of lactate over glucose were calculated. The results are given in the table below.

TABLE 7
Comparison of the production of lactate with strains
overexpressing the yedU gene and with the same
strain without overexpression

| | Lactate titer | Lactate yield |
|---|---|---|
| Strain | (mg/l) | (g/g glucose) |
| Strain 1 | 280 +/− 10 | 0.39 +/− 0.03 |
| | (n = 3) | (n = 3) |
| Strain 2 | 320 +/− 10 | 0.37 +/− 0.02 |
| | (n = 3) | (n = 3) |
| Control strain | 30 +/− 30 | 0.06 +/− 0.11 |
| | (n = 3) | (n = 3) |

n is the number of repetitions of the same experiment.
The figures given are the mean and standard deviation of the figures obtained for n repetitions.

1. An isolated polypeptide having a glyoxalase III enzymatic activity comprising the sequence of SEQ ID NO 1, a fragment or homologous sequence thereof.

2. The polypeptide of claim 1 comprising a sequence having at least 70% identity with the sequence of SEQ ID NO 1.

3. The polypeptide of claim 1 comprising at least 100 contiguous amino acids from the sequence of SEQ ID NO 1.

4. The polypeptide of claim 1 consisting in of the sequence of SEQ ID NO 1.

5. A polynucleotide comprising a sequence coding for the polypeptide of claim 1.

6. The polynucleotide of claim 5 comprising the sequence of SEQ ID NO 2.

7. An expression cassette comprising the polynucleotide of claim 5 under control of regulatory elements functional into a host microorganism.

8. A transformation vector comprising the polynucleotide of one of claim 5.

9. A modified microorganism having modulated glyoxalase III enzymatic activity, wherein activity of the polypeptide of claim 1 is attenuated or enhanced.

10. The microorganism of claim 9, selected among the group consisting of bacteria, yeast and fungi.

11. The microorganism of claim 10, wherein the bacteria is selected among the group consisting of Enterobacteriaceae, Bacillaceae, Streptomycetaceae and Corynebacteriaceae.

12. The microorganism of claim 11, selected among the group consisting of Escherichia coli, Bacillus subtilis, Clostridium acetobutylicum and Corynebacterium glutamicum.

13. The microorganism of claim 9 with attenuated glyoxalase III enzymatic activity wherein expression of native gene coding for the polypeptide is attenuated.

14. The microorganism of claim 13, wherein said microorganism is further modified to enhance production of 1,2-propanediol and/or acetol.

15. The microorganism of claim 14, wherein said microorganism comprises at least one of the following modifications to enhance 1,2-propanediol production and combinations thereof:
Attenuation of the expression of at least one of the following genes: ptsG, ptsH ptsI, crr, edd, eda, gloA, aldA, aldB, ldhA, pflA, pflB, adhE, tpiA, gapA, pykA, pykF, ackA, pta, poxB, arcA and ndh.
Enhancement of the expression of at least one of the following genes: galP, glk, ppsA, mgsA, yqhD, yafB, ydhF, ycdW, yqhE, yeaE, yghZ, yajO, tas, ydjG, ydbC, gldA, fucO.
Modification of the gene lpd such as it has a point mutation leading to a replacement of alanine 55 by valine in the protein encoded by the gene.

16. The microorganism of claim 14, wherein said microorganism comprises at least one of the following modifications to enhance acetol production and a combination thereof:
Attenuation of the expression of at least one of the following genes: ptsG, ptsH, ptsI, crr, edd, eda, gloA, aldA, aldB, ldhA, pflA, pflB, adhE, tpiA, gapA, pykA, pykF, ackA, pta, poxB and gldA.
Enhancement of the expression of at least one of the following genes: galP, glk, ppsA, mgsA, yqhD, yafB, ydhF, ycdW, yqhE, yeaE, yghZ, yajO, tas, ydjG, ydbC.

17. The microorganism of claim 9 with enhanced glyoxalase III enzymatic activity, wherein a polynucleotide is overexpressed.

18. A microorganism transformed with the vector of claim 8.

19. A microorganism wherein the polynucleotide of claim 5 is integrated into a chromosome thereof.

20. A microorganism of claim 9 with enhanced glyoxylase III activity, wherein expression of native gene coding for said polypeptide is enhanced.

21. The microorganism of claim 20, wherein said microorganism comprises a strong promoter upstream the coding sequence of the native gene coding for said polypeptide.

22. The microorganism of claim 17 wherein said microorganism is further modified to enhance production of lactate.

23. The microorganism of claim 22, wherein said microorganism comprises at least one of the following modifications to enhance lactate production and a combination thereof:
Attenuation of the expression of at least one of the following genes: ptsG, ptsH, ptsI, crr, edd, eda, gloA, aldA, aldB, pflA, pflB, frdABCD, adhE, gapA, pykA, pykF, ackA, pta, poxB, yqhD, yafB, ydhF, ycdW, yqhE, yeaE, yghZ, yajO, tas, ydjG, ydbC, gldA, lldD, dld and tpiA
Enhancement of the expression of at least one of the following genes: galP, glk, ppsA and mgsA.

24. A method for modulating the glyoxalase III enzymatic activity in a microorganism, wherein the activity of the polypeptide of claim 1 is enhanced or attenuated in the said microorganism.

25. The method of claim 24, wherein the glyoxalase III enzymatic activity is enhanced by overexpressing a polynucleotide.

26. The method of claim 24, wherein the glyoxalase III enzymatic activity is attenuated by attenuating the expression of a polynucleotide.

27. A method for preparing 1,2-propanediol and/or acetol wherein the microorganism of claim 13 is grown in an appropriate culture medium comprising a source of carbon and the 1,2-propanediol and/or acetol is recovered.

28. The method of claim 27 wherein the recovered 1,2-propanediol and/or acetol is purified.

29. A method for preparing lactate wherein the microorganism of claim 17 is grown in an appropriate culture medium comprising a source of carbon, and the lactate is recovered.

30. The method of claim 29 wherein the recovered lactate is purified.

31. An isolated polypeptide having a glyoxalase III enzymatic activity comprising the sequence of SEQ ID 1, a sequence having at least 70% identity with the sequence of SEQ ID 1, at least 100 contiguous amino acids from the sequence of SEQ ID 1, a fragment of the sequence of SEQ ID 1, and/or a homologue of the sequence of SEQ ID 1.


SEQUENCE LISTING (36 sequences)

SEQ ID NO: 1 - protein, 283 aa, Escherichia coli [sequence omitted]

SEQ ID NO: 2 - DNA, 852 bp, Escherichia coli
atgactgttcaaacaagtaaaaatccgcaggtcgatattgctgaagataatgcattcttcccttcagaatattcgcttagccaatataccagtcctgtctctgatcttgatggcgtggactatccaaaaccttatcgcggtaaacataaaattctggtgatcgccgcggacgaacgttatttgccgaccgataacggaaaactgttctcgaccggtaaccatccgattgaaacgttgctgccgttgtatcatctccatgctgcaggtttcgaattcgaagtggcgaccatttccggtctgatgaccaagtttgaatactgggctatgccgcacaaagatgaaaaagtaatgccattctttgagcagcataaatcgttgttccgcaatccgaagaaactcgcggatgttgttgccagcctcaacgctgatagcgaatatgcagcaatctttgttcctggtggtcatggcgcacttattggtttacctgaaagccaggacgtggctgccgctttacagtgggcaatcaaaaatgaccgttttgttatctccctttgccacggcccggcggcttttctggcgcttcgccacggcgataacccactgaatggttattccatttgcgcattcccagacgccgcagacaaacaaacgccagagattggctatatgccgggtcatctcacctggtacttcggcgaagaactgaagaaaatgggcatgaatatcattaatgacgacatcaccgggcgagtacataaggaccgtaaacttctcaccggcgacagtccttttgcagcgaatgcgttgggtaaactggcggcgcaggaaatgctggcagcttacgcgggttaa

SEQ ID NO: 3 - DNA, 20 bp, Artificial
  Oligonucleotide
ggtgatgatagttatcgccg

SEQ ID NO: 4 - DNA, 20 bp, Artificial
  Oligonucleotide
cgtgccatcgacagcagtcc

SEQ ID NO: 5 - DNA, 30 bp, artificial
  Oligonucleotide
agacattaaaaatatacgtgcagctacccg

SEQ ID NO: 6 - DNA, 30 bp, artificial
  Oligonucleotide
gtgaaagctgacaacccttttgatctttta

SEQ ID NO: 7 - DNA, 22 bp, Artificial
  Oligonucleotide
ggctcattgcaccaccatccag

SEQ ID NO: 8 - DNA, 24 bp, Artificial
  Oligonucleotide
gaaaagacgcgctgacaatacgcc

SEQ ID NO: 9 - DNA, 20 bp, artificial
  Oligonucleotide
gccatcagcaggcttagccg

SEQ ID NO: 10 - DNA, 23 bp, artificial
  Oligonucleotide
gggtattgtggcatgtttaaccg

SEQ ID NO: 11 - DNA, 31 bp, artificial
  Oligonucleotide
gaagtggtcgatgccgggattgaagaatggg

SEQ ID NO: 12 - DNA, 31 bp, artificial
  Oligonucleotide
gggttacgtttcagtgaggcgcgttctgcgg

SEQ ID NO: 13 - DNA, 31 bp, artificial
  Oligonucleotide
tgcagcggcgcacgatggcgacgttccgccg

SEQ ID NO: 14 - DNA, 31 bp, artificial
  Oligonucleotide
cacgatgacgaccattcatgcctatactggc

SEQ ID NO: 15 - DNA, 40 bp, artificial
  Oligonucleotide
catatttccctcaaagaatataaaaaagaacaattaacgc

SEQ ID NO: 16 - DNA, 31 bp, artificial
  Oligonucleotide
tatgttcatgcgatggcgcaccagctgggcg

SEQ ID NO: 17 - DNA, 24 bp, artificial
  Oligonucleotide
ccccggaatcagaggaatagtccc

SEQ ID NO: 18 - DNA, 29 bp, artificial
  Oligonucleotide
gggtagactccattactgaggcgtgggcg

SEQ ID NO: 19 - DNA, 20 bp, Artificial
  Oligonucleotide
gccatcagcaggcttagcgc

SEQ ID NO: 20 - DNA, 23 bp, Artificial
  Oligonucleotide
gggtattgtggcatgtttaaccg

SEQ ID NO: 21 - DNA, 21 bp, Artificial
  Oligonucleotide
ggcacaataacatcaaccagc

SEQ ID NO: 22 - DNA, 22 bp, Artificial
  Oligonucleotide
gccggaaacattgaagagctgg

SEQ ID NO: 23 - DNA, 33 bp, Artificial
  Oligonucleotide
ggcgtctcgccatacaacaaacgcacatcgggc

SEQ ID NO: 24 - DNA, 29 bp, Artificial
  Oligonucleotide
gggctttgccgacaccttcttcgttcttg

SEQ ID NO: 25 - DNA, 100 bp, Artificial
  Oligonucleotide
gaaactcgccgtttatagcacaaaacagtacgacaagaagtacctgcaacaggtgaacgagtcctttggctttgagctggtgtaggctggagctgcttcg

SEQ ID NO: 26 - DNA, 101 bp, Artificial
  Oligonucleotide
ttaaaccagttcgttcgggcaggtttcgcctttttccagattgcttaagttttgcagcgtagtctgagaaatactggtcagcatatgaatatcctccttag

SEQ ID NO: 27 - DNA, 103 bp, Artificial
  Oligonucleotide
atgactgttcaaacaagtaaaaatccgcaggtcgatattgctgaagataatgcattcttcccttcagaatattcgcttagccgtgtaggctggagctgcttcg

SEQ ID NO: 28 - DNA, 101 bp, Artificial
  Oligonucleotide
ttaacccgcgtaagctgccagcatttcctgcgccgccagtttacccaacgcattcgctgcaaaaggactgtcgccggtgagcatatgaatatcctccttag

SEQ ID NO: 29 - DNA, 24 bp, Artificial
  Oligonucleotide
ccgacagtaagacgggtaagcctg

SEQ ID NO: 30 - DNA, 22 bp, Artificial
  Oligonucleotide
agcttagtaaagccctcgctag

SEQ ID NO: 31 - DNA, 34 bp, Artificial
  Oligonucleotide
catgtcatgactgttcaaacaagtaaaaatccgc

SEQ ID NO: 32 - DNA, 28 bp, Artificial
  Oligonucleotide
ctacccgggcatagggcttcagtacgcc

SEQ ID NO: 33 - DNA, 29 bp, Artificial
  Oligonucleotide
caccatgactgttcaaacaagtaaaaatc

SEQ ID NO: 34 - DNA, 21 bp, Artificial
  Oligonucleotide
ttaacccgcgtaagctgccag

SEQ ID NO: 35 - DNA, 34 bp, Artificial; identical to SEQ ID NO: 31
  Oligonucleotide

SEQ ID NO: 36 - DNA, 24 bp, Artificial
  Oligonucleotide
cattcaaacgtaatacgtattaac